(19) United States
(12) Patent Application Publication
(10) Pub. No.: US 2011/0208072 A1
(43) Pub. Date: Aug. 25, 2011

(54) DEVICE FOR DETERMINING THE BLOOD VOLUME AND/OR BLOOD VOLUMETRIC FLOW AND METHOD FOR OPERATING THE SAME

(76) Inventors: Ulrich Pfeiffer, Munich (DE); Reinheld Knoll, Munich (DE)

(21) Appl. No.: 12/996,341

(22) PCT Filed: Jun. 4, 2009
(86) PCT No.: PCT/EP2009/004019

§ 371 Date: Apr. 11, 2011

(30) Foreign Application Priority Data
Jun. 4, 2008 (DE) 10 2008 026 708.2

(51) Int. Cl. A61B 5/026

(52) U.S. Cl. 600/504

(57) ABSTRACT

The invention relates to a device (1) for determining the blood volume and/or blood volumetric flow of a circulation section, comprising a fluid storage unit (2) for storing a measuring fluid (3), an injection device for the injection of the measuring fluid into the circulatory system, at least one pump device (5) disposed between the fluid storage unit and the injection device, means for determining at least one property of the measuring fluid before injection into the circulatory system, further means for determining the respective properties in the circulatory system downstream of the injection site, and an analysis device for calculating the blood volume or volumetric flow as a function of the properties determined. The invention further relates to such a device having means for conditioning the measuring fluid. The invention provides that the measuring fluid can be supplied from the fluid storage unit to a second pump device (9) by means of a first pump device (5), and from the second pump device to the catheter. The invention further provides that the measuring fluid can be supplied to the catheter (11) at a first volumetric flow provided by the first pump device, said first volumetric flow able to be temporarily, in particular regularly, superimposed by a second volumetric flow provided by the second pump device, and/or that the means for conditioning the measuring ring fluid act upon at least one pump device. The invention also relates to a method for operating such devices.

The invention relates to a device for determining the blood volume and/or blood volumetric flow of a circulation section, with a fluid storage unit for storing a measuring fluid, an injection means, especially a catheter (11) or a cannula, for injecting the measuring fluid into the circulatory system, at least one pump device arranged between the fluid storage unit and the injection means, means for determining at least one property of the measuring fluid prior to the injection into the circulatory system, further means for determining the respective property in the circulatory system downstream from the injection site, as well as an analysis device for calculating the blood volume or volumetric flow as a function of the properties determined. The invention moreover relates to such a device with means for conditioning the measuring fluid, as well as a method for operating such devices.

### PRIOR ART

Devices of the kind mentioned above are known from patent application laid-open DE 42 14 068 A1. The devices described therein consist of a fluid reservoir in the form of an infusion pouch, which is filled with a cooled measuring fluid. A suitable measuring fluid can be, for example, an isotonic electrolyte solution (such as table salt solution, Ringer solution, lithium salt solution), an isotonic nonionic solution (e.g., glucose solution) or fatfree nutritional solution, a dye solution (Evans blue, indocyanin green), but also a hypertonic table salt solution. The measuring fluid is supplied via a drip chamber by means of a pump device through a valve device to a catheter, by means of which the measuring fluid is injected in pulsating manner into the patient's blood stream. The inflow of the measuring fluid, which is cooler than body temperature, influences the blood temperature downstream from the injection site.

This method, known as thermodilution, is used, for example, to determine the cardiac output. In this case, a catheter outfitted with several channels, such as that known from U.S. Pat. No. 4,817,624 (e.g., pulmonalis thermodilution infiltration catheter), is placed in the circulatory system such that the cooled measuring fluid is injected through a first channel in front of the right ventricle and the temperature of the blood flow after the right ventricle is determined by means of a temperature sensor arranged on another channel. An analysis device can then calculate the cardiac output (HZV) using the Stewart-Hamilton formula from the volume of the supplied measuring fluid, the temperatures of the supplied injectate and the blood, as well as the temperature variation in the blood stream downstream from the injection site.

We have:

$$H Z V = \frac{{V}_{I} * ( {T}_{B} - {T}_{I} ) * K}{\int ( Δ T * t ) d t}$$

with V_{I}=injectate volume
T_{B}=blood temperature
T_{I}=injectate temperature
K=correction factor for specific gravity and specific heat of injectate and blood
t=time

In addition, with the methods known in practice, one can use the cardiac output and, for example, mean transit time, median transit time, ratio of the steepness of the rising and falling part of the indicator dilution curve, to determine the throughput volume and partial volumes. For a central vein injection of indicator and arterial measurement of indicator, these are for example the intrathoracic blood volume, the global end-diastolic volume, the actual heart filling volume and the extravasal pulmonary water.

An intermittently performed indicator dilution can be used to repeatedly calibrate another continuously performed method of flow and possibly also volume determination. Often this technique is the calibration of the arterial pulse contour method for continuous determination of the cardiac output. But it can also find use in other continuous methods for determining cardiac output, such as transesophageal or transthoracic echo-cardiography, esophageal or external ultrasound Doppler sonography or the electrical thoracic bioimpedance method. As a rule, what is common to all continuous methods is that they do not per se furnish very precise measurement results, but are raised to a substantially higher precision class by frequent recalibration.

Thermodilution can also be measured transpulmonary. As an alternative to the Stewart Hamilton formula, the evaluation can be done by means of the cross correlation after Yelderman, as in U.S. Pat. No. 4,507,974.

According to a first sample embodiment disclosed in DE 42 14 068 A1, the pump device consists of a hose pump, in which the hose line leading from the fluid reservoir to the catheter is compressed consecutively downstream by a cam arrangement. The fluid column located in the hose line is hereupon displaced in the direction of the injection site and goes through a check valve arranged downstream from the pump device, which only opens for a flow from the fluid reservoir to the catheter and prevents a back flowing.

According to another sample embodiment in DE 42 14 068 A1, the pump device is configured as a piston/cylinder arrangement outfitted with a piston drive, which can be connected hydraulically to the fluid reservoir and the catheter via a two-way valve. In a first valve setting, the measuring fluid flows from the fluid reservoir into the piston/cylinder arrangement, while the piston is retracted by the drive unit as the chamber volume increases. After the valve switches, the measuring fluid built up in the piston/cylinder arrangement can be supplied to the catheter, while the piston advances forward as the chamber volume gets smaller.

As regards the procedure of thermodilution and the use of devices of this kind to determine the blood volume or volumetric flow of a circulatory section, express reference is made to the specification in the patent application laid open DE 42 14 068 A1.

To prevent embolisms, an air sensor is arranged in the hose line leading to the catheter downstream from the pump device in both sample embodiments, being connected to the controls of the thermodilution device. Although the infusion pouch can be compressed by suitable means in order to prevent a penetration of air by the excess pressure generated in this way, it cannot be ruled out with certainty that air bubbles may form in the injectate, so that the injection process must be interrupted if this is detected by the air sensor. Furthermore, the possibility exists that the injectate will get heated and thus influence the measurement result on the way from the fluid reservoir to the catheter, especially when it spends time in the piston/cylinder arrangement.

U.S. Pat. No. 5,037,396 discloses an automated injection device for carrying out thermodilution with a thermally insulated, interchangeable piston/cylinder arrangement, whose piston can move thanks to a crank mechanism. The injectate is drawn from the fluid reservoir by the movement of the piston and goes through a heat exchanger cassette, in which the fluid is cooled by means of a thermoelectric temperature control device. The injection of the bolus is done by expressing the measuring fluid from the piston/cylinder arrangement, while a valve device in the already described manner controls the flow both into and out from the piston/cylinder arrangement.

This device has the drawback of a substantial apparatus expense for the cooling of the measuring fluid. Furthermore, it cannot be ruled out that the cooled injectate will be heated to a certain degree during the relatively time-intensive filling and emptying of the piston/cylinder arrangement, despite the insulation.

An injection device for performing a thermodilution with similar structural layout is known from the U.S. Pat. No. 4,502,488, which can be optionally filled by means of an arrangement of several valves from a first reservoir with a first fluid or from a second, temperature-controlled reservoir with a cooled measuring fluid.

Additional devices for quasicontinuous performance of the thermodilution are disclosed in the U.S. Pat. No. 4,507,974 and U.S. Pat. No. 6,736,782, but they have not won acceptance in practice on account of the high expense and for reasons of reliability.

The U.S. Pat. No. 4,212,298 describes a thermodilution injector with a piston/cylinder arrangement, which is pneumatically operated to equalize the injection process.

In the U.S. Pat. No. 5,526,817 and U.S. Pat. No. 4,327,724, moreover, devices are disclosed for manual or partly automated dispensing of the liquid bolus.

In the U.S. Pat. No. 5,676,145 a catheter is described for the performance of thermodilution, in which the bolus of the measuring fluid is pumped by means of an infusion pump outfitted with control and recording means. The catheter furthermore has means of continuous determination of the oxygen saturation of the blood stream.

Moreover, a device is known from U.S. Pat. No. 6,004,275 for performing a thermodilution to determine the cardiac output by the Gorlin formula. The catheter introduced into the heart in this case is provided along its course with a plurality of openings, which are connected to a corresponding number of pressure measuring devices by fluid-filled channels running in the catheter. These detect the pressure in the Arteria pulmonaris, in the right auricle, and in the left auricle. Moreover, the catheter has a temperature sensor, as well as an expandable balloon to narrow the flow cross section.

### Problem

On the one hand, the problem on which the present invention is based is to reliably prevent the penetration of air into the injectate in a device of the kind in question for determination, especially in a repeated and automated fashion, of the blood volume and/or volumetric flow of a circulation section. Furthermore, the device should be suitable to perform measures with therapeutically necessary or at least not contraindicated fluid amounts. Moreover, the problem on which the invention is based is to prevent as much as possible an unwanted influencing of the measurement result by a change in properties of the measuring fluid on its way to the catheter. Furthermore, the attending personnel should be informed promptly as to the performance of the measurement so as to not disturb the measurement process needlessly. In addition, the measurement program of the analysis device should be able to independently recognize erroneous or perturbed dilution measurements and should not display them, being errors, or not use them for recalibration of another continuous method for flow and/or volume determination.

### Solution

The problem on which the invention is based is solved in regard to the formation of air bubbles in a device of the kind in question in that the measuring fluid can be taken from the fluid storage unit by means of a first pump device to a second pump device and from the latter to the blood circulation.

Moreover, a device of the kind mentioned at the outset is suitable for the solving of the problem, in which the measuring fluid can be supplied to the blood circulation with a first volume flow provided by the first pump device, on which a second volume flow provided by the second pump device can be superimposed temporarily, preferably in periodic recurrence.

Thanks to these configurations, the injectate volumetric flow placed in the blood circulation can be modulated. As a rule, no retroactive influence occurs from the measuring and analysis device on the means modulating the volumetric flow. The handling and the safety functions of the first pump device remain unimpaired. Moreover, it is assured that above-atmospheric pressure prevails continuously in the overall infusion system and therefore no air gets into the system, so that as a rule no air detector is needed.

With regard to the unwanted influencing of the measurement result, the solution of the problem of the invention in a device of the aforementioned kind, which is outfitted with means for conditioning the measuring fluid on its way between fluid storage unit and injection means, occurs in that the means for conditioning act at least on one pump device. This guarantees that the measuring fluid still has the most favorable properties for the measurement just prior to the injection.

The informing of the attending personnel as to the upcoming measurement is done according to the invention in that the volumetric flow of the first pump device is determined and the fill volume of the second pump device is ascertained, from this the time at which the second pump device is filled is calculated and a signal is put out in the time interval prior to the activating of the second pump device.

Furthermore, according to one embodiment of the invention, the device is outfitted with an inlet for the body's own blood. Thus, blood is withdrawn from the body of the patient and subsequently used as measuring fluid, and for this purpose it is provided with properties suitable for measurement purposes. Inlet and injection means can be arranged [on?] a device for blood treatment, especially one for dialysis.

The property to be determined in one preferred embodiment of the invention is the temperature of the measuring fluid and that in the blood circulation, since thermodilution is established as a method for determination of the blood volume and blood volumetric flow, especially that of the heart, and the temporary cooling of the blood is reversed with no further ado.

The first pump device is preferably suitable for continuous delivery of fluid and it provides a volumetric flow adequate for the timely filling of the second pump device and/or for longer-lasting injection with low delivery rate. Thanks to its mechanical construction and the safety features, a hose pump is especially suited for this, especially an ordinary infusion pump. As an alternative, one can also use, for example, a pump of an arteriovenous hemodialysis machine, in which case the patient's own blood is constantly circulated and used as measuring fluid.

The second, supporting pump device is preferably provided for discontinuous delivery of fluids and is hydraulically connected to the catheter alternatively or additionally to the first pump device, for modulation of the injectate flow. On account of its simple construction, the second pump device is especially a piston/cylinder arrangement. Alternatively, a membrane pump or a plate compression device with injectate pouch located in between can also be used as the second pump device.

According to a first preferred alternative, the piston of the second pump device can be operationally connected to a drive mechanism, which pushes it uniformly into the cylinder in order to give off a constant volumetric flow.

The drive mechanism here is preferably a pneumatic drive, which acts only on the piston being pushed in, but not on the piston when it is extending. The extending of the piston is accomplished by the delivery pressure of the first pump device.

With special advantage, the piston in a retracted and extended end position acts on end stops, which control the drive mechanism via a control mechanism, such as a bistable 3/2-way valve.

A check valve is arranged between the second pump device and the first pump device and/or advantageously a threshold valve opening in dependence on the pressure downstream from the second pump device. The check valve prevents the measuring fluid from flowing backward when the second pump device is activated, while the threshold valve prevents flow in the direction of the catheter while the second pump device is being filled.

According to another advantageous alternative, the drive mechanism is a force accumulator interacting with the piston, especially a compression spring, in order to lessen the structural expense. Advantageously, a bistable two-way valve with one blocking position and one flow-through position is arranged downstream from the second pump device, which blocks the flow connection to the catheter while the piston/cylinder arrangement is being filled and thereby allows the necessary fluid pressure to be built up to tension the force accumulator. The piston preferably acts on end stops in a retracted and extended end position, which control the two-way valve via a control mechanism.

The means for conditioning the measuring fluid in a second pump device, especially one configured as a piston/cylinder arrangement, are preferably configured as means for temperature control of the measuring fluid, since thermodilution is the preferred measurement method. for this, commercially available Peltier elements are advantageously used, due to their availability and the low acquisition costs.

The devices, moreover, are advantageously designed so that the infusion, injection and hose system carrying the measuring fluid according to the invention, i.e., at least the catheter and hose line, including branches leading away from bifurcations, is fashioned as a onetime-use sterile system of tolerated materials, especially polyurethane or polyethylene or polycarbonate.

### FIGURES

The figures show various embodiments of the invention schematically and as examples. The indicated parameters refer to an adult of normal weight (body weight 70 kg).

There are shown:

FIG. 1: a first device according to the invention for determination of the blood volume or volumetric flow of a circulation section in the starting position;

FIG. 2: the same device after filling of the second pump device;

FIG. 3: the device of FIGS. 1 and 2 after emptying of the second pump device;

FIG. 4: another device according to the invention;

FIG. 5: a device according to another embodiment of the invention in the starting position;

FIG. 6: the same device after filling of the second pump device;

FIG. 7: the device of FIGS. 5 and 6 after emptying of the second pump device;

FIG. 8: a device according to another preferred embodiment.

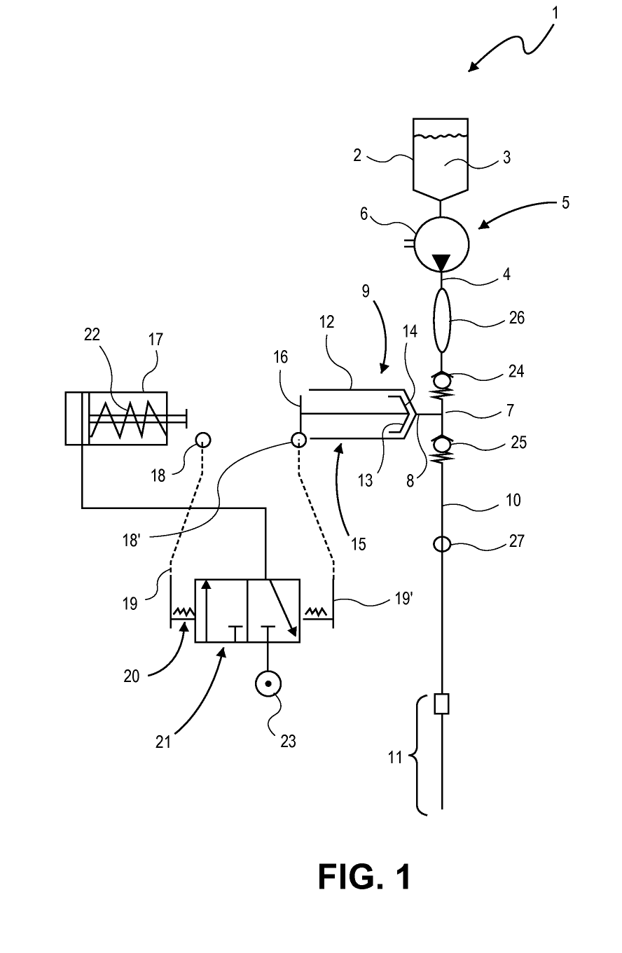

The device 1 represented in FIG. 1 for determination of the blood volume or volumetric flow of a circulation section has a fluid storage unit 2, such as an infusion pouch or a bottlelike container, in which the measuring fluid 3 is stockpiled. Such a measuring fluid can be, for example, an indicator fluid. The measuring fluid 3 can be provided with properties that form the basis for the later measurement process already in the fluid storage unit 2. In the sample embodiment, the measuring fluid 3 for a thermodilution is already provided in the cooled state, i.e., with a temperature below body temperature.

The entire infusion, injection and hose system carrying the measuring fluid 3 is preferably fashioned as a onetime-use sterile system of tolerated materials, especially polyurethane or polyethylene.

The measuring fluid is drawn out from the fluid storage unit 2 across a hose line 4 by means of a first pump device 5 in the form of an infusion pump 6 from the fluid storage unit 2 and delivered in the direction of a bifurcation 7 in the hose line 4, from which a first branch 8 is connected to a second pump device 9 and the second branch 10 is connected to the catheter 11 introduced in the patient's blood stream.

The second pump device 9 is syringe-like in design and consists of a cylinder 12, in which a piston 13 is movably arranged. The branch 8 emerges at the end face in the chamber 14 of the piston/cylinder arrangement 15, which can change its volume by the displacement of the piston 13. The overhanging end 16 of the piston 13 in its extended end position impinges on a pneumatic drive mechanism 17 located in its initial position and on an end stop 18, which is operationally connected via a mechanical connection 19 to the adjusting mechanism 20 of a pneumatic 3/2-way valve 21. In the retracted end position of the piston 13, another end stop 18′ is activated, which is connected by a mechanical connection 19′ likewise to the bistable adjustment mechanism 20 of the 3/2-way valve 21. Instead of the end stops 18, 18′ and mechanical connections 19, 19′, one can basically also use electrical or pneumatic switches or conduits, respectively. The initialization of the pneumatic drive 17 takes place under the action of a spring 22, as soon as the 3/2-way valve 21 in its venting position shown in FIG. 1 cuts off the drive 17 from the pressurized air port 23. The pressurized air port 23 can be connected, for example, to the pressurized air network of a hospital or a separate pressurized air generator.

Upstream from the bifurcation 7, a check valve 24 is arranged in the hose line 4, which blocks the back flow directed opposite the delivery flow of the first pump device 5. Downstream from the bifurcation 7, in the branch 10, there is furthermore a threshold valve 25 that opens in dependence on the pressure.

Insofar as a repeat measurement is involved, the piston 12 of the piston/cylinder arrangement 15 is situated in its maximum retracted position, as shown in FIG. 1, at the start of the preparing of the injection process, where the smallest possible volume of the chamber 14 occurs. The infusion pump 6 is adjusted to a relatively small, but constant volume flow V1, which can be, for example, between 12.5 ml/h (=300 ml/24 h) and 50 ml/h (=1200 ml/24 h). In the sample embodiment, the delivery volume is set at 25 ml/h. When the infusion pump 6 is started, the measuring fluid 3 delivered by it is injected through the branch 8 of the hose line 4 into the chamber 14 of the piston/cylinder arrangement 15, under opening of the check valve 24, since the branch 10 of the hose line 4 leading from the bifurcation 7 in the direction of the catheter 11 is blocked by the threshold valve 25. The frictional forces to be overcome in the displacement of the piston 13 are designed to be so low that the pressure created in the measuring fluid 3 upstream from the threshold valve 25 lies below the opening pressure of the threshold valve 25.

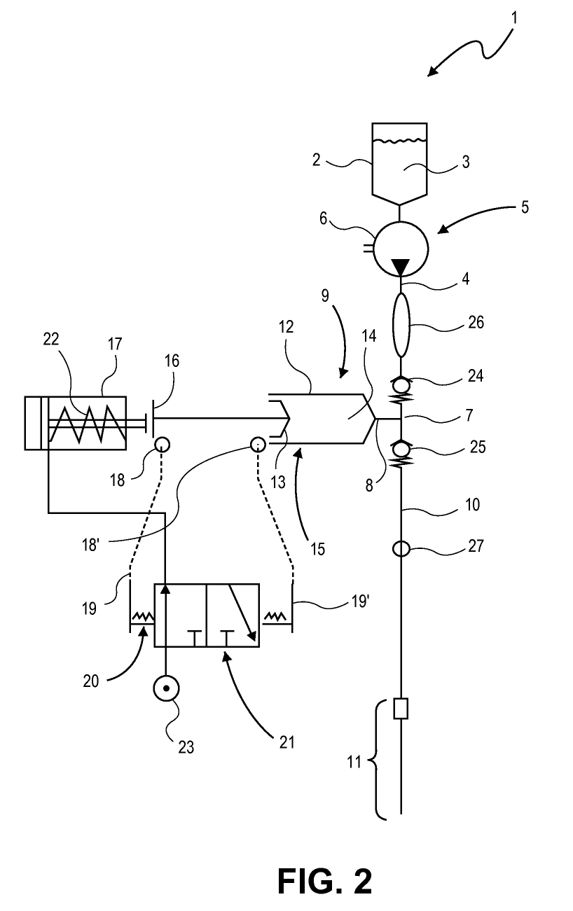

As the chamber 14 is filled to its maximum capacity of 25 ml in the sample embodiment, the piston 13 is extended from the cylinder 12 until its overhanging end 16 acts against the end stop 18 (FIG. 2). The volume of measuring fluid 3 now contained in the chamber 14 is preferably between 10 and 25 ml. In the sample embodiment, this is set at 25 ml, i.e., the maximum capacity of the chamber 14.

The device 1 is outfitted with an acoustic warning system, which indicates the upcoming thermodilution with a waiting time of around 2 minutes. Actions which might lead to a falsification of the measurement result by influencing the blood temperature should then be halted.

By activating the stop 18, the 3/2-way valve 21 is moved by means of the bistable adjusting mechanism 20 to its second position, in which the pressurized air port 23 is connected to the pneumatic drive 17 and lets it work against the extended piston 13 of the second pump device 9.

The pneumatic drive 17 is designed so that the full volume of the chamber 14 is expelled from the piston/cylinder arrangement 15 at a constant and relatively high volume flow V2 within 1 to 10 seconds. Under the acting of the delivery pressure that builds up, the check valve 24 closes, so that a return flow in the direction of the fluid storage unit 2 is prevented. An elastically expandable region 26 in the hose line 4 receives the measuring fluid still delivered by the injection pump 6 upstream from the check valve 24. The elasticity of the region 26 is chosen such that the pressure in the hose line 4 between check valve 24 and first pump device 5 lies definitely below the shut-off pressure of the infusion pump 6.

At the same time, the threshold valve 25 opens and clears the flow channel between the second pump device 9 and the catheter 11. The volume flow V3 injected through the catheter 11 corresponds to the volume flow V2 injected by the second pump device 9. The threshold valve 25 is preferably outfitted with a device for measuring the central venous blood pressure. After the opening of the threshold valve 25, a measurable pressure peak or rather long-lasting rise in pressure occurs, whose rising edge designates the start of the injection process and can be used to activate the thermodilution analysis device. Furthermore, the temperature of the measuring fluid 3 flowing into the catheter 11 is detected by means of a temperature sensor 27 in the branch 10 of the hose line 4. The course of the blood temperature is memorized for a period of 10 to 30 seconds.

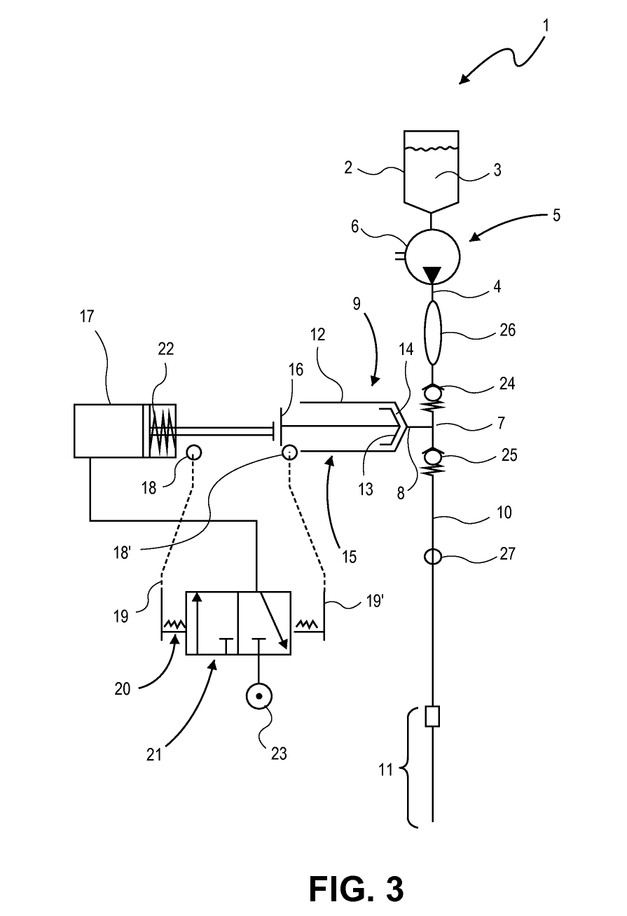

Once the desired injection volume has been expelled from the piston/cylinder arrangement 15, the overhanging end 16 of the retracting piston 13 impinges on the second end stop 18′ with the result that the 3/2-way valve 21 once again cuts off the pneumatic drive 17 from the pressurized air port 23 and provides venting (FIG. 3). The pneumatic drive 17 subsequently travels back to its starting position per FIG. 1 under the action of the spring 22. At the same time, the pressure in the region of the bifurcation 7 decreases, so that the threshold valve 25 again closes. The pressure drop, moreover, characterizes the end of the measurement process, so that the necessary duration of the measurement for the analysis can be determined. The delivery pressure of the infusion pump 6 is subsequently enough to open the check valve 24. As the elastic region 26 of the hose line is emptied, a renewed filling of the piston/cylinder arrangement 15 now occurs.

Calculation and graph plotting of the measurement results occur automatically by the analysis unit, not shown. Furthermore, with the end of the measurement, which can be determined by the pressure drop in the system, a message is sent that the restrictions regarding the influencing of the blood temperature are lifted until the waiting time for the next measurement. Measurements are discarded if, for example, the blood temperature baseline prior to the injection drifts above a predetermined threshold value, such as +/−0.1°/minute or the signal/noise ratio (S/N) is <10. Moreover, the thermodilution curve should have certain criteria. Thus, for example, the rise should be steeper than the fall. Moreover, the fall should basically be monoexponential. Perturbations or peaks in the curve can likewise indicate an invalid measurement.

If one knows the volume flow V1 delivered by the infusion pump 6 and the capacity of the piston/cylinder arrangement 15, the cycle times for consecutively occurring measurements can be calculated. Normally, the piston/cylinder arrangement 15 is already partly filled before the first measurement is triggered, for example, with a volume of 24 ml. When the device 1 is placed in use, the volume of 1 ml needed for complete filling of the chamber 14 first has to be injected into the piston/cylinder arrangement 15. For the volume flow V1 of 25 ml/h adjusted at the infusion pump 6, this occurs within 2.4 minutes (=2 min, 24 s). Taking into account the delivery tolerances of the infusion pump 6 of +/−5%, one gets a time window of 2.28 minutes (=2 min, 16.8 s) to 2.52 minutes (=2 min, 31.2 s). Furthermore, the elasticity of the hose line system should be taken into account, since a portion of the measuring fluid delivered by the infusion pump 6 is taken up by the hose line 4 through elastic expansion. This dead volume is device-specific and can therefore be determined in advance and taken into account as a system constant in the calculation. If the dead volume in the sample embodiment is 1 ml, this leads to a doubling of the time needed to prepare for the first measurement, i.e., between 4.56 minutes and 5.04 minutes. For the second and following measurements, the chamber 14 has to be filled completely each time, i.e., with 25 ml. Taking into account the dead volume of the hose line 4, we thus have a cycle time between 57 minutes and 63 minutes. The cycle times determined in this way can be used to put out the above described warnings of upcoming measurements.

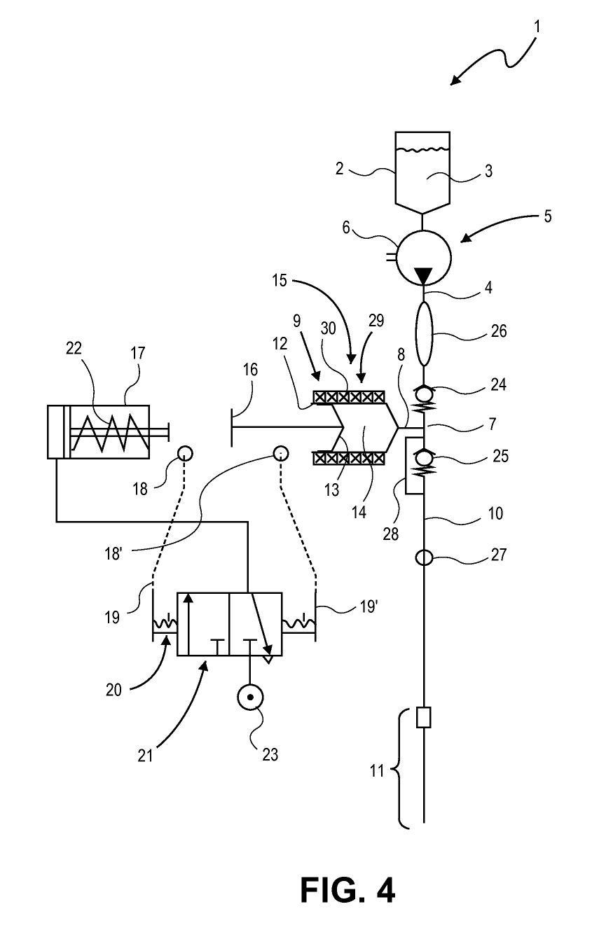

The sample embodiment shown in FIG. 4 basically corresponds to the device of FIGS. 1 to 3. However, the threshold valve 25 is coordinated with a capillary with defined fluid throughput, acting as a bypass 28, by which a partial flow V1 of the volume flow V1 to the catheter delivered by the first pump device 5 also flows during the filling of the second pump device 9. Whereas in the first sample embodiment a cycle is formed with one phase of high injection throughput and one phase without injection, by using the bypass 28 one can achieve an alternation of high and lower throughput.

The piston/cylinder arrangement 15 is moreover outfitted with means 29 of temperature control of the measuring fluid 3, by which a conditioning is brought about during the time spent in the second pump device 9. The means 29 consist of Peltier elements 30, which surround the outer lateral surface of the cylinder 12 and cool it down after an electrical potential is applied.

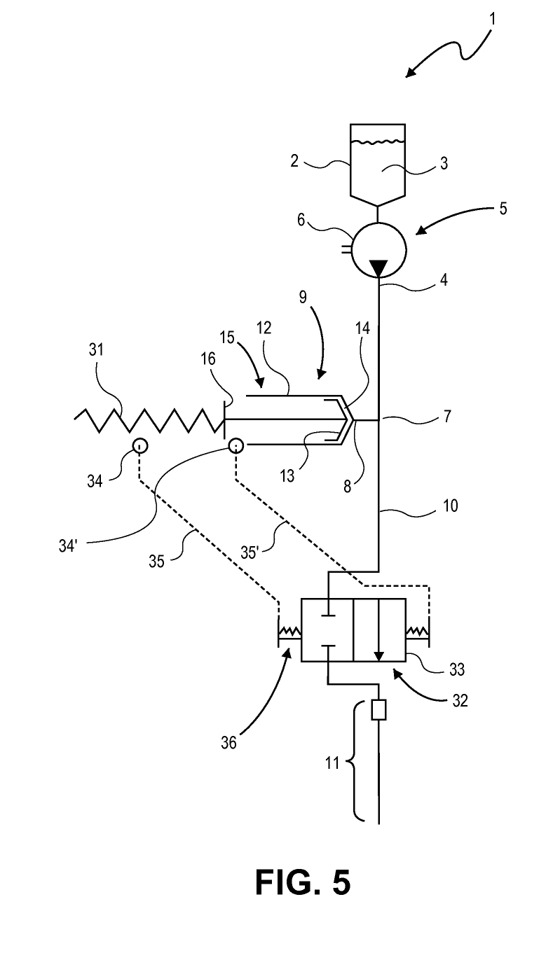

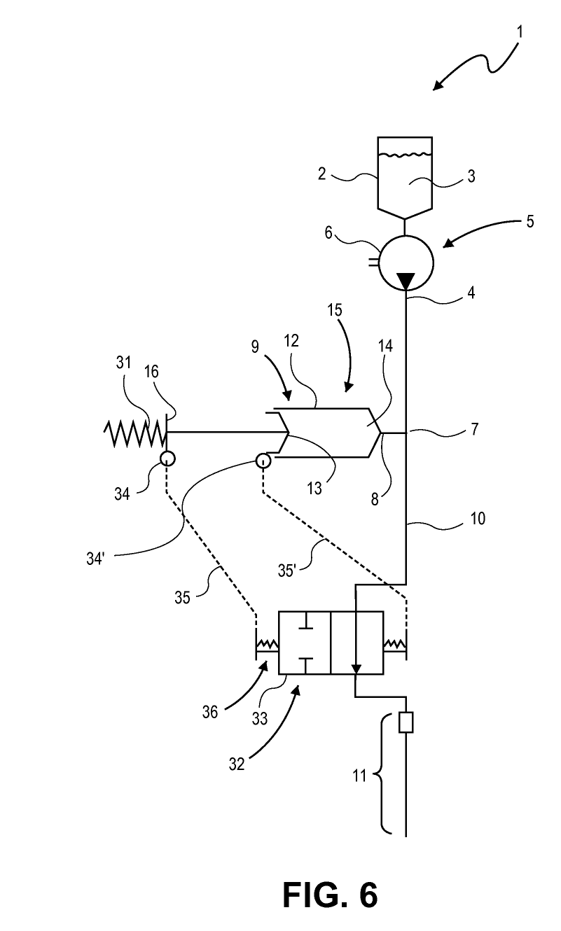

In the device 1 shown in FIGS. 5 to 7, the piston 13 of the piston/cylinder arrangement 15 acts directly against a helical compression spring 31, which is tensioned by the piston 13 extending as measuring fluid 3 is injected into the chamber 14. In the hose line 4 downstream from the bifurcation 7 there is a valve arrangement in the branch 10 in the form of a two-way valve 33, which has a blocking position and a through position. When preparing for the measurement process, the two-way valve 33 is placed in the blocking position (FIG. 5). The volume flow V1 delivered by the infusion pump 6 is therefore diverted from the bifurcation 7 across the branch 8 into the chamber 14 of the piston/cylinder arrangement 15, which is filled in this way. The piston 13 that is forced out in this way, when the desired full volume is reached, acts against an end stop 34, which is joined by a mechanical connection 35 to the adjustment device 36 of the mechanically operated bistable two-way valve 33. The two-way valve 33 is in this way moved into the through position, so that the flow channel between the bifurcation 7 and the catheter 11 is opened up (FIG. 6).

Thanks to this movement of the valve device 32, the constant volume flow V1 of measuring fluid 3 that is delivered by the still-operating infusion pump 6 can flow through the valve 23 into the catheter 11. On this is superimposed a second volume flow V2(t), which is injected back into the hose line 4 by the piston/cylinder arrangement 15 across the branch 8, while the pressure spring 31 forces the piston 13 into the cylinder 12 as the volume of the chamber 14 gets smaller. The second volume flow V2(t) decreases slightly with time, since the pressing force created by the compression spring 31 diminishes somewhat during the retraction of the piston 13 on account of lessening of the spring force. Thus, over a period of 1 to 10 seconds, there occurs at first a phase in which a high volume flow V3(t)=V2(t)+V1, yet one diminishing over time, is injected across the catheter 11 into the blood stream of the patient. Thanks to a suitably dimensioned compression spring 31, a volume flow V3(t) deviating by no more than 20% from the rectangular flow pattern is preferably selected. The volume of measuring fluid 3 injected by the piston/cylinder arrangement 15 is known and corresponds to the capacity of the chamber 14, plus the measuring fluid delivered by the infusion pump during the injection time.

The maximum delivery pressure of the first pump device 5 is apportioned so that it lies above the highest activation pressure of the second pump device 9. In theory, the second pump device 9 cannot furnish any pressure higher than the first pump device 5. Thus, no measuring fluid 3 can flow from the piston/cylinder arrangement 15 back to the fluid storage unit opposite the delivery direction of the infusion pump 6. The delivery pressure of the infusion pump 6 must be sufficient to extend the piston 13 of the piston/cylinder arrangement 15 against the force of the compression spring 31.

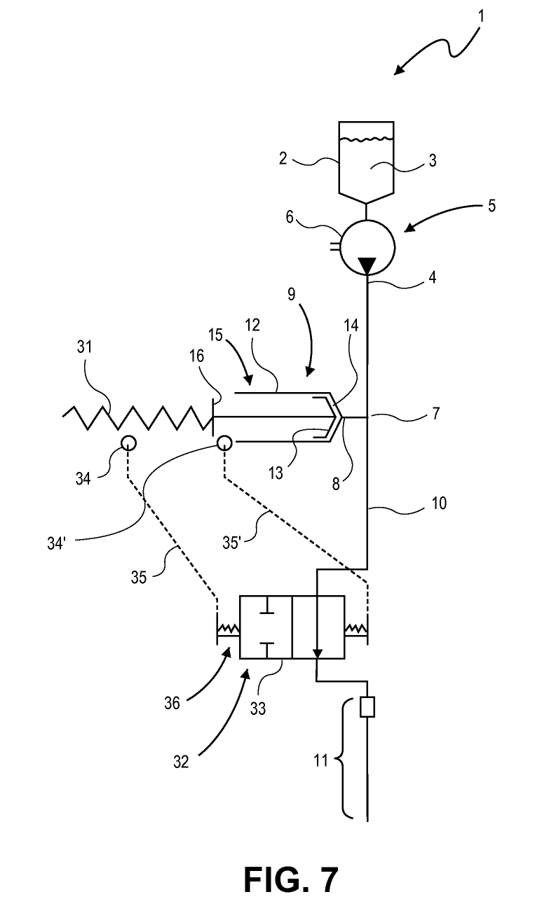

After the emptying of the chamber 14 (FIG. 7), the piston 13 retracted into the cylinder 12 now acts against another end stop 34′. This activates the adjustment mechanism 36 of the two-way valve 33, which resets the valve mechanism 32 to the initial position per FIG. 5.

Instead of the end stops 34, 34 and mechanical connections 35, 35′, one can basically also use electrical or pneumatic switches or conduits, respectively.

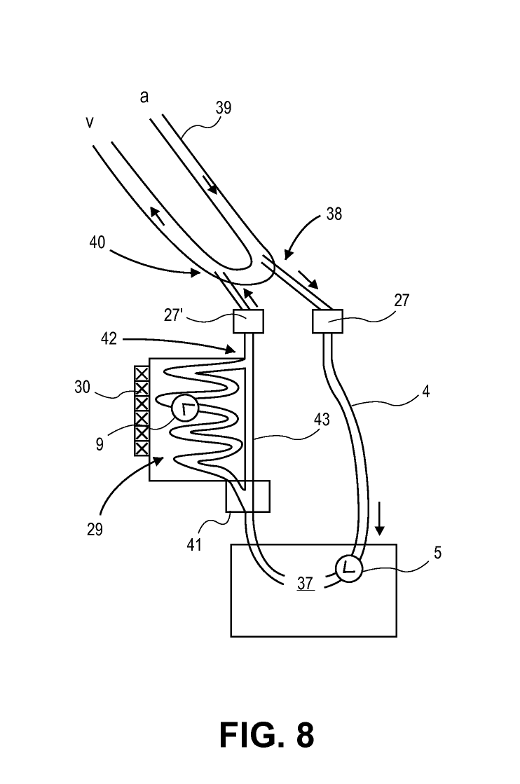

In the embodiment shown in FIG. 8, the device of the invention is operationally connected to a blood treatment device in the form of a dialysis machine 37, which is outfitted with a pump device 5 for essentially continuous delivery of a blood flow. The dialysis machine 37 is hydraulically connected by an inlet 38 to the vascular system 39 of the dialysis patient. In the region of the inlet 38, a first temperature sensor 27 is arranged in the hose system 4, which measures the temperature of the arterial blood withdrawn from the blood circulation. Another temperature sensor 27′ is located immediately upstream from the venous shunt cannula 40. Between the dialysis machine 37 and the temperature sensor 27′, moreover, a means 29 is provided for temperature control of the blood removed from the blood circulation, which is cooled there by means of Peltier elements 30, for example. The means 29 for temperature control is hydraulically connected across an actively controlled Y-valve 41 and a junction 42, situated downstream therefrom, to the hose line 4, which forms a bypass 43 between Y-valve 41 and junction 42.

To prepare the measurement, blood in an amount sufficient for thermodilution is injected into the means 29 for temperature control by switching of the Y-valve 41, while the bypass 43 is blocked. Next, the Y-valve 41 is switched back, so that the blood emerging from the dialysis machine 37 flows back into the vascular system 39 across the bypass 43. After a sufficient cooling of the previously stockpiled blood, another switching of the Y-valve 41 takes place. Now, the blood flow delivered by the pump device 5 drives the cooled blood, serving as measuring fluid 3, in the direction of the venous shunt cannula 40, passing by the temperature sensor 27′.

A second, supporting pump device 9 can be arranged in the region of the means 29 for temperature control, which speeds up the injection of the cooled blood. As a rule, however, this will not be required, since 20 ml of blood cooled in a typical dialysis pump flow of 300 mg/min and a reservoir yields a sufficiently short injection time of 4 seconds.

The dialysis hose system, including the temperature sensors 27, 27′, the Y-valve 41, the bypass 43 and the pouchlike means 29 with a meandering blood passage is preferably made as a disposable article of tolerated materials, such as polyurethane, polyethylene or polycarbonate.

In the sample embodiments, the measurement of the blood volume or volumetric flow is done by tracking the time variation of the blood temperature after the injecting of a cooled fluid bolus. However, informative dilution curves can also be obtained essentially by determining the effects of other properties of measuring fluids, such as deliberate altering of the oxygen saturation in the blood stream or the injecting of salts, dyes, or radioactively labeled substances. However, thermodilution is the customary technique, since the cooling of the blood flow produced by the injectate bolus is limited in time and restores itself without further ado.

Moreover, for example, by introducing a controllable blocking or releasing control valve into the branch 8 in place of the rigid time model for the phase of high through flow or a variable delay in the adjustment mechanism 20 or 36, one can also generate a complex pattern, such as a pseudorandom one.

piston/cylinder arrangement 15

### LIST OF REFERENCE NUMBERS

1 device (for determination of the blood volume or volumetric flow of a circulation section)
2 fluid storage unit
3 measuring fluid
4 hose line
5 first pump device
6 infusion pump
7 bifurcation
8 branch
9 second pump device
10 branch
11 catheter
12 cylinder
13 piston
14 chamber
15 piston/cylinder arrangement
16 end (of piston)
17 drive (pneumatic)
18, 18′ end stop
19,19′ connection
20 adjustment mechanism
21 2-way valve
22 spring
23 pressurized air port
24 check valve
25 threshold valve
26 region (elastic)
27, 27′ temperature sensor
28 bypass
29 means (for temperature control)
30 Peltier element
31 compression spring
32 valve mechanism
33 2-way valve
34, 34′ end stop
35, 35′ connection
36 adjustment mechanism
37 dialysis machine
38 inlet
39 vascular system
40 shunt cannula
41 Y-valve
42 junction
43 bypass
V₁ volume flow (in first pump device)
V₂ volume flow (in second pump device)
V₃ in catheter

1. Device for determining the blood volume and/or blood volumetric flow of a circulation section, with
a fluid storage unit (2) for storing a measuring fluid (3),
an injection means, especially a catheter (11) or a cannula, for injecting the measuring fluid (3) into the circulatory system,
at least one pump device (5) arranged between the fluid storage unit (2) and the injection
means,
means for determining at least one property of the measuring fluid (3) prior to the injection into the circulatory system,
means for determining the respective property in the circulatory system downstream from the injection site, and
an analysis device for calculating the blood volume or volumetric flow as a function of the properties determined,
characterized in that
the measuring fluid (3) can be taken from the fluid storage unit (2) by means of a first pump device (5) to a second pump device (9) and from the second pump device (9) to the blood circulation.

2. Device (1) for determining the blood volume and/or blood volumetric flow of a circulation section, with
a fluid storage unit (2) for storing a measuring fluid (3),
an injection means, especially a catheter (11) or a cannula, for injecting the measuring fluid (3) into the circulatory system,
at least one pump device (5) arranged between the fluid storage unit (2) and the injection means,
means for determining at least one property of the measuring fluid (3) prior to the injection into the circulatory system,
means for determining the respective property in the circulatory system downstream from the injection site, and
an analysis device for calculating the blood volume or volumetric flow as a function of the properties determined,
especially according to claim 1,
characterized in that
the measuring fluid (3) can be supplied to the blood circulation with a first volume flow (V1) provided by a first pump device (5), on which a second volume flow (V2) provided by a second pump device (9) can be superimposed temporarily.

3. Device according to claim 2, characterized in that the temporary superimposing is periodically repeatable.

4. Device according to one of claims 1 to 3, characterized in that the property to be determined is the temperature of the measuring fluid (3) and that in the blood circulation.

5. Device according to one of claims 1 to 4, characterized in that the first pump device (5) is suitable for continuous delivery of the measuring fluid (3).

6. Device according to claim 5, characterized in that the first pump device (5) is a hose pump, especially an infusion pump (6).

7. Device according to one of the preceding claims, characterized in that the second pump device (9) is preferably provided for discontinuous delivery of the measuring fluid (3).

8. Device according to claim 7, characterized in that the second pump device (9) is a piston/cylinder arrangement (15) or a membrane pump.

9. Device according to claim 8, characterized in that the piston (13) of the second pump device (9) can be operationally connected to a drive mechanism.

10. Device according to claim 9, characterized in that the drive mechanism is a pneumatic drive (17), which acts only on the piston (13) being pushed in.

11. Device according to claim 9 or 10, characterized in that the piston (13) in a retracted and extended end position acts on end stops (18, 18′), which control the drive mechanism via a control mechanism.

12. Device according to one of claims 9 to 11, characterized in that a check valve (24) is arranged between the second pump device (9) and the first pump device (5) and/or a threshold valve (25) opening in dependence on the pressure is arranged downstream from the second pump device.

13. Device according to claim 9, characterized in that the drive mechanism is a force accumulator interacting with the piston (13), especially a compression spring (31).

14. Device according to claim 13, characterized in that a two-way valve (33) with one blocking position and one flow-through position is arranged downstream from the second pump device (9).

15. Device according to claim 13 or 14, characterized in that the piston (13) acts on end stops (34, 34′) in a retracted and extended end position, which control the two-way valve (33) via a control mechanism.

16. Method for operating the device according to one of the preceding claims, characterized in that
the volumetric flow (V1) of the first pump device (5) is determined and the fill volume of the second pump device (9) is ascertained,
next the time at which the second pump device (9) is filled is calculated and
a signal is put out in the time interval prior to the activating of the second pump device (9).

17. Device (1) for determining the blood volume and/or blood volumetric flow of a circulation section, with
a fluid storage unit (2) for storing a measuring fluid (3),
an injection means, especially a catheter (11) or a cannula, for injecting the measuring fluid (3) into the circulatory system,
at least one pump device (5, 9) arranged between the fluid storage unit (2) and the injection means,
means for determining at least one property of the measuring fluid (3) prior to the injection into the circulatory system,
means for determining at least one property in the circulatory system downstream from the injection site, and
an analysis device for calculating the blood volume or volumetric flow as a function of the properties determined,
wherein the device is outfitted with means for conditioning the measuring fluid on its way between fluid storage unit (2) and injection means,
especially according to one of claims 1 to 15,
characterized in that
the means for conditioning act on at least one pump device (5, 9).

18. Device according to claim 17, characterized in that the means for conditioning are configured as means (29) for temperature control of the measuring fluid (3).

19. Device according to claim 17 or 18, characterized in that the conditionable pump device (9) is configured as a piston/cylinder arrangement (15) or membrane pump.

20. Device according to one of claims 17 to 19, characterized in that the means (29) for temperature control comprise Peltier elements (30).

21. Device according to one of claims 1 to 15 and 17 to 20, characterized in that of the structural parts carrying the measuring fluid (3) at least the catheter (11) and hose line (4) are fashioned as a onetime-use sterile system of tolerated materials, especially polyurethane or polyethylene.

22. Device (1) for determining the blood volume and/or blood volumetric flow of a circulation section, with
a hose line (4) for delivering a measuring fluid (3),
an injection means, especially a catheter (11) or a cannula, for injecting the measuring fluid (3) into the circulatory system,
at least one pump device (5, 9) arranged downstream from the injection means,
means for determining at least one property of the measuring fluid (3) prior to the injection into the circulatory system,
means for determining at least one property in the circulatory system downstream from the injection site, and
an analysis device for calculating the blood volume or volumetric flow as a function of the properties determined,
characterized in that
the device is outfitted with an inlet for the body's own blood.

23. Device according to claim 22, characterized in that a device for blood treatment, especially one for dialysis, is arranged between inlet and injection means.
